(19) United States
(12) Patent Application Publication
(10) Pub. No.: US 2023/0390197 A1
(43) Pub. Date: Dec. 7, 2023

(54) MODULAR DENDRON MICELLES FOR COMBINATION IMMUNOTHERAPY

(71) Applicant: Wisconsin Alumni Research Foundation, Madison, WI (US)

(72) Inventors: Seungpyo Hong, Madison, WI (US); Deric L. Wheeler, Blue Mounds, WI (US)

(21) Appl. No.: 18/250,065

(22) PCT Filed: Oct. 26, 2021
(86) PCT No.: PCT/US2021/056547

§ 371 Date: Apr. 21, 2023

Related U.S. Application Data
(60) Provisional application No. 63/106,070, filed on Oct. 27, 2020.

(51) Int. Cl. A61K 9/107; A61K 47/34; A61K 47/10; A61K 47/69; A61K 47/59; A61K 47/60; A61P 35/00; A61K 31/337

(52) U.S. Cl. CPC A61K 9/1075; A61K 47/34; A61K 47/10; A61K 47/6907; A61K 47/593; A61K 47/60; A61P 35/00; A61K 31/337

(57) ABSTRACT

A self-assembled immunotherapeutic dendron-micelle includes a first amphiphilic dendron-coil, a second amphiphilic dendron-coil, and a third amphiphilic dendron-coil. The first and second amphiphilic dendron-coils have immunotherapeutic peptides conjugated thereto. Also included are pharmaceutical compositions containing the dendron-micelles, methods of making the dendron-micelles, and immunotherapy methods including administering the dendron-micelles to a subject in need thereof.

### CROSS-REFERENCE TO RELATED APPLICATIONS

This application claims priority to U.S. Provisional Application 63/106,070 filed on Oct. 27, 2020, which is incorporated herein by reference in its entirety.

### FIELD OF THE DISCLOSURE

The present disclosure is related to a drug delivery system for combination immunotherapy.

### BACKGROUND

Breast cancer is the most commonly diagnosed cancer among American women (>260,000 cases annually), and one of the leading causes of cancer death (>40,000/year) in the U.S. In particular, triple negative breast cancer (TNBC) represents approximately 10-15% of all breast cancers, which is typically associated with a poorer clinical outcome than other subtypes of breast cancer. Paclitaxel is one of the commonly used adjuvant chemotherapeutic drugs against TNBC, and the emerging immunotherapy targeting immune checkpoints, such as programmed death-ligand 1 (PD-L1) expressed on tumor cells, has shown promising results in clinical trials. However, a major hurdle of both therapies is their lack of, or limited at best, specificity toward tumor cells, causing undesirable side effects.

Head and Neck Squamous Cell Carcinoma (HNSCC) is the sixth most common cancer worldwide with over 600,000 new cases diagnosed annually. Standard of care treatments for HNSCC patients include surgery, radiation and chemotherapy. Additionally, the anti-epidermal growth factor receptor (EGFR) monoclonal antibody cetuximab (CTX) is often used in combination with these treatment modalities, and the emerging programed cell death protein 1 (PD1)/PD-ligand 1 (PD-L1) checkpoint inhibitors, nivolumab and pembrolizumab, are now approved in the metastatic setting. Despite clinical response with these therapeutics, it remains a challenge to appropriately deliver these treatments to the targeted cell.

What is needed are novel methods and combinations for delivering chemotherapeutic agents to treat cancers such as TNBC and HNSCC.

### BRIEF SUMMARY

In an aspect, a self-assembled immunotherapeutic dendron-micelle comprises a first amphiphilic dendron-coil, a second amphiphilic dendron-coil, and a third amphiphilic dendron-coil; wherein the first amphiphilic dendron-coil comprises a first non-peptidyl, hydrophobic core-forming component covalently linked to a first polyester dendron which is covalently linked to first a poly(ethylene glycol) (PEG) moiety, wherein the first PEG moiety comprises a first conjugated immunotherapeutic peptide; wherein the second amphiphilic dendron-coil comprises a second non-peptidyl, hydrophobic core-forming component covalently linked to a second polyester dendron which is covalently linked to a second poly(ethylene glycol) (PEG) moiety, wherein the second PEG moiety comprises a second conjugated immunotherapeutic peptide; and wherein the third amphiphilic dendron-coil comprises a third non-peptidyl, hydrophobic core-forming component covalently linked to a third polyester dendron which is covalently linked to a third poly(ethylene glycol) (PEG) moiety, wherein the third PEG moiety does not comprise a conjugated immunotherapeutic peptide.

In another aspect, a pharmaceutical composition comprises the self-assembled immunotherapeutic dendron-micelle described above.

Also included are immunotherapy methods comprising administering a therapeutically effective amount of the self-assembled immunotherapeutic dendron-micelle described above.

In another aspect, a method of making a self-assembled immunotherapeutic dendron-micelle comprises synthesizing a first amphiphilic dendron-coil by covalently linking a first non-peptidyl, hydrophobic core-forming component to a first polyester dendron, covalently linking the first polyester dendron to a first poly(ethylene glycol) (PEG) moiety, and conjugating a first therapeutic peptide to the first PEG moiety; synthesizing a second amphiphilic dendron-coil by covalently linking a second non-peptidyl, hydrophobic core-forming component to a second polyester dendron, covalently linking the second polyester dendron to a second poly(ethylene glycol) (PEG) moiety, and conjugating a second therapeutic peptide to the second PEG moiety; synthesizing a third amphiphilic dendron-coil by covalently linking a third non-peptidyl, hydrophobic core-forming component to a third polyester dendron, covalently linking the third polyester dendron to a third poly(ethylene glycol) (PEG) moiety, wherein the third PEG moiety does not comprise a conjugated immunotherapeutic peptide; and incubating the first, second, and third amphiphilic dendron-coils under conditions for self-assembly of the self-assembled immunotherapeutic dendron-micelle.

### BRIEF DESCRIPTION OF THE DRAWINGS

FIG. 1. is a schematic diagram of preparation of a DM from PDCs synthesized through click chemistry between PCL and G3 dendron, followed by PEGylation conjugation.

FIG. 2 shows the anticipated targeting and therapeutic action of the proposed multifunctional dendron micelles: i) EGFR-targeting; ii) PD-L1 targeting and immune checkpoint blockade; and iii) paclitaxel release to the tumor cells.

FIG. 3A-D show multi-step synthesis of dendron-based block copolymers. 3A. Chemical scheme of conjugation of PCL, G3 dendron, and PEG; 3B. ¹H-NMR spectra confirming each synthetic steps; 3C. GPC chromatograms showing increases in molecular weight upon conjugation of each polymer; and 3D. FT-IR spectra confirming the PCL-dendron conjugation by observing disappearance of the azide group at ˜2,200 cm-1.

FIG. 4 shows a linear relationship between CMC and HLB for (●) PDCs and (▪) linear-block copolymers.

FIG. 5A-B simulated structures using molecular dynamics. 5A shows the dendron micelle structure formed from PDC. 5B. micelle assembled from linear copolymer.

FIG. 6 shows drug release profiles of various DMs containing indomethacin. Note that drug release becomes slower with an increase in molecular weight of PCL block.

FIG. 7 shows dissociation constants (KD) of free aPD-L1 vs. dendrimer-aPD-L1 conjugates. SPR, surface plasmon resonance; BLI, biolayer interferometry (BLI); AFM, atomic force microscopy.

FIG. 8 shows IL-2 secretion from Jurkat T cells upon incubation with tumor cells treated with free dendrimers (G7), free antibody (aPD-L1), and G7-aPD-L1 conjugates.

FIG. 9 shows balb/c mice bearing 4T1 xenograft after treated with A) G7-aPD-L1, B) G7-IgG, and C) free aPD-L1. The white arrows indicate the tumor sites. D) Quantitative analysis of the amount of each material at the tumor sites, showing the >4-fold higher accumulation of G7-aPD-L1 compared to free aPD-L1. * denotes p=0.025.

FIG. 10 shows In vitro and in vivo tests of G7-aEGFR conjugates vs. free dendrimers: MDA-MB-468 cells treated with A) G7-aEGFR and B) free dendrimers; C57BL/6 mice bearing MOC1 xenograft after injected with C) G7-aEGFR and D) free G7.

FIG. 11A-C show various assay results comparing peptides and antibodies: 11A) SPR sensograms of dendrimer-pPD-1 (G7-βH2_mt), free aPDL1, and free pPD-1 (βH2_mt). The dendrimer-peptide conjugates exhibit comparable KD to the whole antibody (aPD-L1), which is 5 orders of magnitude stronger than free peptide. 11B) The dendrimer-peptide conjugates exhibiting specific interaction with high PD-L1 expressing 786O cells (bottom image). 11C) Cell retention assay from the surface immobilized with aEGFR, EG1, EG2, and EG1/2 mixture. Note that the EG1/2 mixture exhibits comparable cell retention to that of aEGFR.

FIG. 12 shows a mix-and-match approach for preparation of a variety of multifunctional dendron micelles. DM1 (EGFR-targeting DM w/paclitaxel), DM2 (PD-L1-targeting DM w/paclitaxel), and DM3 (dual targeted DM w/paclitaxel) will be prepared and assessed.

FIG. 13 shows synthetic routes of various PDCs functionalized with various bioactive molecules.

FIG. 14 shows MALDI-MSI technology for distinguishing differences in lipid expression following drug exposure in rat brain.

FIG. 15 shows a schematic illustration of the planned in vivo experiments.

The above-described and other features will be appreciated and understood by those skilled in the art from the following detailed description, drawings, and appended claims.

### DETAILED DESCRIPTION

Described herein is a combination delivery system that incorporates two or more immunotherapeutic peptides and optionally a drug such as a chemotherapeutic or anti-inflammatory drug. Considering the side effects from therapies that are not specific to cancer cells, a drug delivery system that can integrate cancer targeting, immunotherapy, and chemotherapy, for example, is a promising approach to substantially increase the treatment efficacy. To address this need, the modular delivery system described herein can carry immunotherapeutic and chemotherapeutic drugs specifically to cancer cells, to maximize the therapeutic efficacy while minimizing toxic bystander effects. To realize this goal, the nanoparticle system described herein contains therapeutic peptides (peptides that are selective to tumor cells and or immune cells) and optionally drug cargo (agents that boost up the immune system against tumor and/or chemotherapeutic drugs). The nanoparticle system is based on a hyperbranched dendron, linear hydrophobic polymer, and poly(ethylene glycol) (PEG) corona.

Triple negative breast cancer (TNBC) represents approximately 10-15% of all breast cancers, which is typically associated with poorer clinical outcomes than other subtypes of breast cancer. Paclitaxel is one of the commonly used adjuvant chemotherapeutic drugs against TNBC, and the emerging immunotherapy targeting immune checkpoints, such as programmed death-ligand 1 (PD-L1) expressed on tumor cells, has shown promising results in clinical trials. Furthermore, the epidermal growth factor receptor (EGFR), which is overexpressed by the majority of TNBC, represents a unique opportunity for specific delivery of such therapeutic agents using nanocarriers. However, the integration of EGFR targeting, PD-L1 inhibition, and chemotherapy remains elusive. To address this, described herein is a novel nanoscale delivery system to concurrently deliver chemotherapeutic and immunotherapeutic agents by bispecifically targeting EGFR- and PD-L1-overexpressing TNBC cells.

Head and Neck Squamous Cell Carcinoma (HNSCC) is the sixth most common cancer worldwide with over 600,000 new cases diagnosed annually. The anti-epidermal growth factor receptor (EGFR) monoclonal antibody cetuximab (CTX) is often used in combination with conventional treatment modalities (chemotherapeutic drug), and emerging immunotherapy, such as the PD-1/PD-L1 checkpoint inhibitor nivolumab, is now approved in the metastatic setting. Despite clinical response with these therapeutics, HNSCC remains difficult to be effectively treated. To synergistically utilize these treatment options, described herein is a nanoscale delivery system to concurrently deliver chemotherapeutic and immunotherapeutic agents by bispecifically targeting EGFR and PD-L1-overexpressing HNSCC cells. The facile integration of the multiple agents will be achieved through dendron micelles (DMs) that can be multifunctionalized through a mix-and-match approach.

Specifically, described herein is the facile integration of the multiple agents through dendron micelles (DMs) that can be modularly multifunctionalized via a mix-and-match approach. The proposed DM system will be prepared through self-assembly of PEGyated dendron copolymers (e.g., PEG-polyester dendron poly-e-caprolactone, or PDCs). The PEGyated dendron copolymers are conjugated with therapeutic peptides such as: i) PD-1 mimicking peptides (pPD-1) and/or ii) EGF-mimicking peptides (EG1 and EG2), along with an optional chemotherapeutic drug (e.g., paclitaxel or docetaxel) encapsulated in the hydrophobic core. The nanoscale delivery system can concurrently deliver chemotherapeutic and immunotherapeutic agents by bispecifically targeting EGFR- and PD-L1—overexpressing TNBC cells in a highly specific manner.

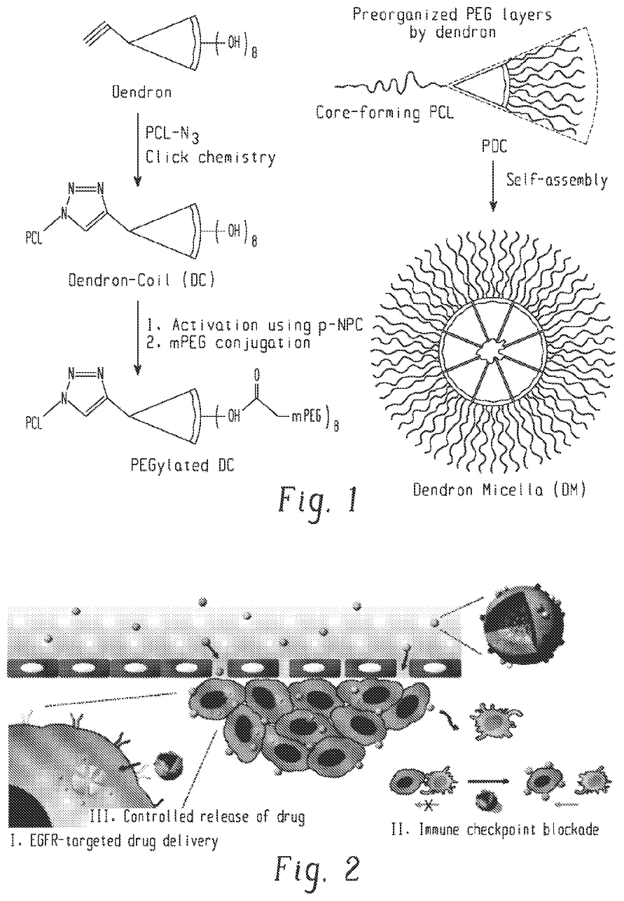

Preparation of a dendron micelle synthesized through click chemistry and the proposed targeting and therapeutic action of the multifunctional dendron micelles are illustrated in FIGS. 1 and 2. First, the pre-organized conical structure imposed by a dendron enables self-assembly with an excellent thermodynamic stability, due to the minimal (pre-paid) entropy cost. Second, the hyperbranched structure of dendrons facilitates the multivalent binding effect that dramatically improves binding kinetics of targeting ligands (e.g. peptides to the level of (or higher than) the binding strength of corresponding antibodies). Third, the high-density poly(ethylene glycol) (PEG) outer layer provides maximal stealth effect for longer plasma circulation as well as modular control over PEG configuration effects on cell interactions. Lastly, the biodegradable, biocompatible polymer components (the core-forming poly-e-caprolactone (PCL) and polyester dendron) allow the controlled release of the drug molecules encapsulated in the core of DMs and mitigate the toxicity concerns. These characteristics of DMs will directly affect the functionality of the molecules integrated within the micelles. The thermodynamic stability will improve the structural integrity of DMs during circulation upon injection. The multivalent binding will maximize the binding kinetics of EGFR-binding peptides and PD-L1-binding peptides (FIG. 1(i) and (ii)), which will substantially increase the targeting efficacy and inhibition of binding between PD-1 and PD-L 1 . The controlled biodegradation will allow paclitaxel to be slowly released over a long period of time (FIG. 2 (iii)).

Advantageously, by incorporating the therapeutic peptides in the self-assembled dendron micelles, the spatial distance among different peptides can be maintained, which will facilitate the binding to different targets on cells, maximizing their biological effects (e.g., immunotherapy efficacy or specificity towards diseased cells).

Described herein are self-assembled dendron micelles comprising two or more chemically distinct amphiphilic dendron-coils (DCs). Each amphiphilic dendron-coil comprises a non-peptidyl, hydrophobic core-forming component which is covalently linked to a polyester dendron which is covalently linked to a poly(ethylene glycol) (PEG) moiety. The hydrophobic core-forming component of the dendron-coils is non-peptidyl, that is, the hydrophobic core-forming block is not a peptide. In an aspect, the PEG moiety of the DC is conjugated to a therapeutic peptide, such as a β-hairpin peptide. Thus, in an aspect, each of the chemically distinct DCs of the self-assembled dendron micelles comprises a different conjugated therapeutic peptide, such as a β-hairpin peptide.

In an aspect, a self-assembled immunotherapeutic dendron-micelle comprises a first amphiphilic dendron-coil, a second amphiphilic dendron-coil, and a third amphiphilic dendron-coil; wherein the first amphiphilic dendron-coil comprises a first non-peptidyl, hydrophobic core-forming component covalently linked to a first polyester dendron which is covalently linked to first a poly(ethylene glycol) (PEG) moiety, wherein the first PEG moiety comprises a first conjugated immunotherapeutic peptide; wherein the second amphiphilic dendron-coil comprises a second non-peptidyl, hydrophobic core-forming component covalently linked to a second polyester dendron which is covalently linked to a second poly(ethylene glycol) (PEG) moiety, wherein the second PEG moiety comprises a second conjugated immunotherapeutic peptide; and wherein the third amphiphilic dendron-coil comprises a third non-peptidyl, hydrophobic core-forming component covalently linked to a third polyester dendron which is covalently linked to a third poly(ethylene glycol) (PEG) moiety, wherein the third PEG moiety does not comprise a conjugated immunotherapeutic peptide.

In an aspect, the first and second immunotherapeutic peptides are different peptides, but can bind the same cellular target. In an aspect, the first and second immunotherapeutic peptides each bind a different cellular target.

In an aspect, the third amphiphilic dendron-coil can comprise a drug, ligand, or label as described herein.

In an aspect, the first and second amphiphilic dendron-coils comprising immunotherapeutic peptides comprise 5 to 80 wt % of the self-assembled immunotherapeutic dendron-micelle, while the third amphiphilic dendron-coil comprises 20 to 95 wt % of the self-assembled immunotherapeutic dendron-micelle. The third amphiphilic dendron-coil with no conjugated peptide provides the basal structure of the micelle and provides spacing between the immunotherapeutic peptides which can improve the efficacy of the micelles.

In an aspect, the self-assembled immunotherapeutic dendron-micelle further comprises a fourth amphiphilic dendron-coil comprising a fourth non-peptidyl, hydrophobic core-forming component covalently linked to a fourth polyester dendron which is covalently linked to a fourth poly(ethylene glycol) (PEG) moiety, wherein the fourth PEG moiety comprises a third conjugated immunotherapeutic peptide, an imaging contrast agent, or a chemotherapeutic or immunotherapeutic drug.

Exemplary non-peptidyl, hydrophobic core-forming components of the DCs comprise polycaprolactone (PCL), poly(lactic acid) (PLA), poly(glycolic acid) (PGA), poly(lactic-co-glycolic acid) (PLGA), or a combination thereof. In an aspect, the non-peptidyl, hydrophobic core-forming component is PCL, such as poly(ε-caprolactone). In an aspect, the non-peptidyl, hydrophobic core-forming component has a molecular weight of about 0.5 kDa to about 20 kDa. In specific aspects, the non-peptidyl, hydrophobic core-forming component is poly(ε-caprolactone) with a molecular weight of about 3.5 kDa or poly(ε-caprolactone) with a molecular weight of 14 kDa.

Exemplary polyester dendrons of the amphiphilic dendron-coil include, but are not limited to, a generation 3 to generation 5 [that is, a generation 3 (G3), a generation 4 (G4) or a generation 5 (G5)] polyester dendron with either an acetylene or carboxylate core. In a specific aspect, the polyester dendron is generation 3 polyester-8-hydroxyl-1-acetylene bis-MPA dendron. Methods of preparing and characterizing dendrons are well known in the art, and various polyester dendrons may be purchased from commercial entities.

Exemplary PEG moieties of the amphiphilic dendron-coil include a methoxy PEG (mPEG) moiety, amine-terminated PEG (PEG-NH₂) moiety, acetylated PEG (PEG-Ac) moiety, carboxylated PEG (PEG-COOH) moiety, thiol-terminated PEG (PEG-SH) moiety, N-hydroxysuccinimide-activated PEG (PEG-NHS) moiety, NH₂-PEG-NH₂ moiety, and an NH₂-PEG-COOH moiety. In aspects, the PEG moiety has a molecular weight including, but not limited to, a molecular weight of about 0.2 kDa to about 5 kDa. In some embodiments, the PEG moiety is an mPEG moiety with a molecular weight of about 2 kDa. In specific aspects, the PEG moiety is an mPEG moiety with a molecular weight of about 5 kDa.

In an aspect, the first conjugated immunotherapeutic peptide binds a first cell-expressed receptor and the second conjugated immunotherapeutic peptide binds a second cell-expressed receptor, wherein the first and second cell-expressed receptors are on the same or different types of target cells for the immunotherapeutic dendron-micelle. For example, one therapeutic peptide can target a T-cell expressed receptor and the other therapeutic peptide can target a tumor cell expressed receptor.

In an aspect, the therapeutic peptide comprises a peptide with high affinity for an immune checkpoint receptors, a growth factor receptor, a cell surface receptor, an intracellular receptor, or the extracellular matrix.

Exemplary immune checkpoint receptors include PD-L1, PD-1, OX40, TIGIT, CTLA-4, CD137 (4-1BB), CD28, and CD27.

Immune checkpoint inhibitor β-hairpin peptides can be identified by identifying immune checkpoint inhibitor ligand peptides, e.g., surface peptides that interact with high affinity with the immune checkpoint receptor surface. For example, surface β-hairpin PD-1 peptides which interact with PD-L1 with high affinity have been identified herein. As used herein, high affinity means K_{D} of 0.1-1,000 nM. Such peptides can have lengths of 5 to 50 amino acids, and do not correspond to the entire immune checkpoint inhibitor.

Exemplary β-hairpin PD-1 peptides include:
SEQ ID NO: 1: TYLCGAISLAPKLQIKESLRA (βH₁-wt sequence)
SEQ ID NO: 2: TYVCGVISLAPRIQIKESLRA (βH₁-mutant sequence)
SEQ ID NO: 3: VLNWYRMSPSNQTDRKAA (βH₂-wt sequence)
SEQ ID NO: 4: HVVWHRESPSGQTDTKAA (βH₂-mutant sequence)

In an aspect, the therapeutic peptide binds a growth factor receptor. Exemplary growth factor receptors include epidermal growth factor receptor (EGFR), insulin-like growth factor receptor (IGFR), transforming growth factor-beta receptor (TGF-βR), human epidermal growth factor receptor 2 (HER2), vascular endothelial growth factor receptor (VEGFR), platelet-derived growth factor receptor (PDGFR), and fibroblast growth factor receptor (FGFR).

The epidermal growth factor receptor (EGFR) family encompasses four receptor proteins, namely ErbB-1/EGFR-1 to -4 (also called HER 1-4) that are expressed on cell surface and exhibit tyrosine kinase activities. These proteins have similar structures and are comprised of three domains: an extracellular domain with ligand binding site, a transmembrane domain, and an intracellular domain with kinase activity.

The insulin-like growth factor receptor (IGFR) family consists of two cell membrane receptors, IGF1R and IGF2R. IGF1R (that also forms a heterodimer with the insulin receptor [IR]) binds to insulin-like growth factor 1 (IGF1) with higher affinity and IGF2 with comparatively lower affinity to elicit the growth signals required for foetal and postnatal development.

The transforming growth factor-beta receptor (TGF-βR) family comprises three membrane receptors (TβRI, TβRII and TβRIII) which are expressed in diverse types of cells and regulate distinct cellular functions by the signals transduced upon TGF-β ligand binding.

Human epidermal growth factor receptor 2 (HER2) receptors plays a central role in the pathogenesis of several human cancers. They regulate cell growth, survival, and differentiation via multiple signal transduction pathways and participate in cellular proliferation and differentiation. The family is made up of four main members: HER-1, HER-2, HER-3, and HER-4, also called ErbB1, ErbB2, ErbB3, and ErbB4.

The vascular endothelial growth factor receptor (VEGFR) family consists of three membrane receptors (VEGFR1-3), predominantly expressed on endothelial cells and few additional cell types. VEGFRs are single pass protein with seven immunoglobulin (Ig)-like domains on the extracellular site and two split tyrosine kinase domains in the intracellular site.

The platelet-derived growth factor receptor (PDGFR) family contains two receptors (PDGFR-α and-β) that are encoded by two different genes and are expressed on the membrane of different cell types. These single chain receptor proteins have five Ig-like extracellular domains and a tyrosine kinase domain.

The fibroblast growth factor receptor (FGFR) family consists of four closely related transmembrane proteins (FGFR1-4) and their different isoforms with altered ligand specificity due to differential splicing of FGFR mRNA. These single chain receptors contain one extracellular domain with three immunoglobulin repeats (Ig I-III) with ligand binding capacity, one transmembrane domain and one intracellular domain with kinase activity at the carboxy-terminus.

Exemplary therapeutic peptides that are tumor targeting peptides that bind cell surface receptors include peptides that bind integrins such as αvβ3 integrin which has an RGD binding motif, and αvβ6 integrin which is expressed on the surface of colon, liver, ovarian, pancreatic, and squamous cancer cells. Additional targets for tumor targeting peptides include aminopeptidase N, peptide transporter 1, epidermal growth factor receptors, prostate-specific membrane antigen, mucinl , urokinase plasminogen activator receptor, gastric-releasing peptide receptor, somatostatin receptor, cholecystokinin receptor, neurotensin receptor, transferrin receptor, vascular endothelial growth factor receptor, insulin, ephrin receptor, and the like.

Therapeutic peptides that bind intracellular receptors include peptides that bind BCR/ABL, a pathogenic fusion protein that is responsible for the chronic phase of chronic myelogenous leukemia (CML), cyclin A, CDK, mitochondria, and the like.

Therapeutic peptides that target the extracellular matrix include peptides that bind fibronectin, a fibroblast growth factor, a matrix metalloproteinase, a prostate-specific antigen, a cathepsis, and the like.

In an aspect, the micelles encapsulate or, in other words, are loaded with one or more drugs. Drugs include cancer drugs (i.e., a drug used to treat cancer), also called chemotherapeutic agents. In some embodiments, the drug is an anti-inflammatory drug including, but not limited to, indomethacin.

A “drug” is a compound that, upon administration to a patient (including, but not limited to, a human or other animal) in a therapeutically effective amount, provides a therapeutic benefit to the patient.

In an aspect, the therapeutic agent is a chemotherapeutic agent. Chemotherapeutic agents include, but are not limited to, the following classes: alkylating agents, antimetabolites, anthracyclines, plant alkaloids, topoisomerase inhibitors, monoclonal antibodies, and other anti-tumor agents. In addition to the chemotherapeutic drugs described above, namely doxorubicin, paclitaxel, other suitable chemotherapy drugs include tyrosine kinase inhibitor imatinib mesylate (Gleeve® or Glivec®), cisplatin, carboplatin, oxaliplatin, mechloethamine, cyclophosphamide, chlorambucil, azathioprine, mercaptopurine, pyrimidine, vincristine, vinblastine, vinorelbine, vindesine, podophyllotoxin (L01CB), etoposide, docetaxel, topoisomerase inhibitors (L01CB and L01XX) irinotecan, topotecan, amsacrine, etoposide, etoposide phosphate, teniposide, dactinomycin, lonidamine, and monoclonal antibodies, such as trastuzumab (Herceptin®), cetuximab, bevacizumab and rituximab (Rituxan®), among others.

The amount of drug present in the micelle can vary over a wide range. The drug can be about 1% to about 30% (weight/weight) of the total mass of the micelle (wherein the mass of the drug is included in the total mass of the micelle). In some aspects, the drug can be about 2% to about 25% w/w of the total mass of the micelle (same basis). In some aspects, the drug can be about 3% to about 20% w/w of the total mass of the micelle (same basis).

In an aspect, the micelles further comprise one or more ligands conjugated to one or more PEG moieties. An exemplary ligand is folic acid.

The term “ligand” refers to a compound that exhibits binding selectivity for a particular target organ, tissue or cell. In an aspect, the ligand binds a cancer cell. One example of a ligand is the vitamin folic acid (FA), which binds folate receptors that are overexpressed in approximately 90% of human ovarian carcinomas. Luteinizing hormone-releasing hormone (LHRH) is another exemplary ligand. LHRH is relatively small molecule (MW 1,182 Da), with the receptors overexpressed by breast, ovarian, and prostate cancer cells. Another exemplary ligand is a retinoid such as retinol, retinal, retinoic acid, rexinoid, or derivatives or analogs thereof. Additional ligands include, but are not limited to, transferrin, RGD peptide, Herceptin, prostate-specific membrane antigen (PSMA)-targeting aptamers, follicle stimulating hormone (FSH), epidermal growth factor (EGF) and the like. Other ligands include various antibodies such as anti-CD19, anti-CD20, anti-CD24, anti-CD33, anti-CD44, Lewis-Y antibody, sialyl Lewis X antibody, LFA-1 antibody, rituximab, bevacizumab, anti-VEGF mAb, and their fragments, dimers, and other modified forms. In other aspects, the ligand targets an immune cell. For targeting immune cells, the ligand can be a ligand of e.g., a T cell surface receptor. Lectins can be used as ligands to target mucin and the mucosal cell layer. Lectins include those isolated from Abrus precatroius, Agaricus bisporus, Glycine max, Lysopersicon esculentum, Mycoplasma gallisepticum, and Naja mocambique, as well as lectins such as Concanavalin A and Succinyl-Concanavalin A.

In an aspect, the ligand increases the selective delivery of the micelle to a particular target organ, tissue or cell. Target organs may include, for example, the liver, pancreas, kidney, lung, esophagus, larynx, bone marrow, and brain. In some aspects, the increase in selective delivery may be at least about two-fold as compared to that of an otherwise comparable composition lacking the targeting agent. In some aspects, the delivery of the micelle containing a ligand to the target organ, tissue or cell is increased by at least 10% or 25% compared to that of an otherwise comparable composition lacking the ligand.

The amount of ligand present in a micelle can vary over a wide range. In some aspects, the ligand can be about 1% to about 80% (weight/weight), specifically about 10% to about 50% w/w, and more specifically be about 20% to about 40% w/w of the total mass of the micelle (wherein the mass of the ligand is included in the total mass of the nanocore).

In aspects, the ligand may be conjugated to the micelle through a covalent bond to PEG. A variety of mechanisms known in the art can be used to form the covalent bond between the ligands and PEG, e.g., a condensation reaction. Additional methods for directly bonding one or more ligands to PEG are known in the art. Chemistries include, but are not limited to, thioether, thioester, malimide and thiol, amine-carboxyl, amine-amine, and others listed in organic chemistry manuals. Ligands can also be attached to PEG using a crosslinking reagent [e.g., glutaraldehyde (GAD), bifunctional oxirane (OXR), ethylene glycol diglycidyl ether (EGDE), N-hydroxysuccinimide (NHS), and a water soluble carbodiimide, such as 1-ethyl-3-(3-dimethylaminopropyl) carbodiimide (EDC)]. The compositions herein can further have at least one hydrolysable linker between the therapeutic agent and scaffold and/or targeting agent and scaffold.

In an aspect, the micelles can include one or more imaging agents or radiosensitizing molecules. Non-limiting examples of paramagnetic ions of potential use as imaging agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III). Radioisotopes of potential use as imaging or therapeutic agents include astatine²¹¹, carbon¹⁴, chromium⁵¹, chlorine³⁶, cobalt⁵⁷, cobalt⁵⁸, copper⁵², copper⁶⁴, copper⁶⁷, fluorine¹⁸, gallium⁶⁷, gallium⁶⁸, hydrogen³, iodine¹²³, iodine¹²⁴, iodine¹²⁵, iodine¹³¹, indium¹¹¹, iron⁵², iron⁵⁹, lutetium¹⁷⁷, phosphorus³², phosphorus³³, rhenium¹⁸⁶, rhenium¹⁸⁸, and selenium⁷⁵ I¹²⁵ is used in some embodiments, and indium¹¹¹ is also used in some embodiments due to its low energy and suitability for long-range detection.

In some aspects, the imaging agent is a secondary binding ligand or an enzyme (an enzyme tag) that will generate a colored product upon contact with a chromogenic substrate. Examples of enzymes include urease, alkaline phosphatase, (horseradish) hydrogen peroxidase and glucose oxidase. Secondary binding ligands are biotin and avidin or streptavidin compounds. The use of such labels is well known in the art.

In some aspects, the imaging agent is a fluorescent label. Non-limiting examples of photodetectable labels include ALEXA FLUORO® 350, ALEXA FLUORO® 430, AMCA, aminoacridine, BODIPY 630/650, BODIPY 650/665, BODIPY-FL, BODIPY-R6G, BODIPY-TMR, BODIPY-TR, 5-carboxy-4¹, 5′-dichloro-2¹, 7¹-dimethoxy fluorescein, 5-carboxy-2′,4′,5′,7′-tetrachlorofluorescein, 5-carboxyfluorescein, 5-carboxyrhodamine, 6-carboxyrhodamine, 6-carboxytetramethyl amino, Cascade Blue, Cy2, Cy3, Cy5,6-FA, dansyl chloride, Fluorescein, HEX, 6-JOE, NBD (7-nitrobenz-2-oxa-1,3-diazole), Oregon Green 488, Oregon Green 500, Oregon Green 514, Pacific Blue, phthalic acid, terephthalic acid, isophthalic acid, cresyl fast violet, cresyl blue violet, brilliant cresyl blue, para-aminobenzoic acid, erythrosine, phthalocyanines, azomethines, cyanines, xanthines, succinylfluoresceins, rare earth metal cryptates, europium trisbipyridine diamine, a europium cryptate or chelate, diamine, dicyanins, La Jolla blue dye, allopycocyanin, allococyanin B, phycocyanin C, phycocyanin R, thiamine, phycoerythrocyanin, phycoerythrin R, REG, Rhodamine Green, rhodamine isothiocyanate, Rhodamine Red, ROX, TAMRA, TET, TRIT (tetramethyl rhodamine isothiol), Tetramethylrhodamine, Edans and TEXAS RED. These and other luminescent labels may be obtained from commercial sources such as Molecular Probes (Eugene, Oreg.), and EMD Biosciences (San Diego, Calif.).

Chemiluminescent agents include luminol, isoluminol, an aromatic acridinium ester, an imidazole, an acridinium salt and an oxalate ester, or a bioluminescent compound such as luciferin, luciferase and aequorin. Diagnostic conjugates may be used, for example, in intraoperative, endoscopic, or intravascular tumor or disease diagnosis.

In some aspects, the outer surface of the micelle is modified. One example of such a modification is modification of the outer surface of the micelle with a long-circulating agent, e.g., glycosaminoglycans. Examples of glycosaminoglycans include hyaluronic acid. The micelles may also, or alternatively, be modified with a cryoprotectant, e.g., a sugar, such as trehalose, sucrose, mannose, glucose or HA. The term “cryoprotectant” refers to an agent that protects a lipid particle subjected to dehydration-rehydration, freeze-thawing, or lyophilization-rehydration from vesicle fusion and/or leakage of vesicle contents.

Also included are pharmaceutical compositions comprising the micelles described herein.

As used herein, “pharmaceutical composition” means therapeutically effective amounts of the nanoparticles together with a pharmaceutically acceptable excipient, such as diluents, preservatives, solubilizers, emulsifiers, and adjuvants. As used herein “pharmaceutically acceptable excipients” are well known to those skilled in the art.

Tablets and capsules for oral administration may be in unit dose form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavoring or coloring agents.

For topical application to the skin, the micelles may be made up into a cream, lotion or ointment. Cream or ointment formulations which may be used for the micelles are conventional formulations well known in the art. Topical administration includes transdermal formulations such as patches.

For topical application to the eye, the micelles may be made up into a solution or suspension in a suitable sterile aqueous or non-aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The micelles may also be administered parenterally in a sterile medium, either subcutaneously, or intravenously, or intramuscularly, or intrasternally, or by infusion techniques, in the form of sterile injectable aqueous or oleaginous suspensions. Depending on the vehicle and concentration used, the micelles can be suspended in the vehicle. Advantageously, adjuvants such as a local anesthetics, preservative and buffering agents can be dissolved in the vehicle.

Pharmaceutical compositions may conveniently be presented in unit dosage form and may be prepared by any of the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

In an aspect, a method of making a self-assembled immunotherapeutic dendron-micelle comprises synthesizing a first amphiphilic dendron-coil by covalently linking a first non-peptidyl, hydrophobic core-forming component to a first polyester dendron, covalently linking the first polyester dendron to a first poly(ethylene glycol) (PEG) moiety, and conjugating a first therapeutic peptide to the first PEG moiety; synthesizing a second amphiphilic dendron-coil by covalently linking a second non-peptidyl, hydrophobic core-forming component to a second polyester dendron, covalently linking the second polyester dendron to a second poly(ethylene glycol) (PEG) moiety, and conjugating a second therapeutic peptide to the second PEG moiety; synthesizing a third amphiphilic dendron-coil by covalently linking a third non-peptidyl, hydrophobic core-forming component to a third polyester dendron, covalently linking the third polyester dendron to a third poly(ethylene glycol) (PEG) moiety, wherein the third PEG moiety does not comprise a conjugated immunotherapeutic peptide; and incubating the first, second, and third amphiphilic dendron-coils under conditions for self-assembly of the self-assembled immunotherapeutic dendron-micelle.

In an aspect, the first, second and/or third amphiphilic dendron-coils can be synthesized using click chemistry between the hydrophobic core-forming component and the polyester dendron. Other art-known chemistries may also be used.

In an aspect, conjugating the first and second therapeutic peptide comprises NH₂-PEG-tBOC conjugation, followed by deprotection with TFA. Other art-known chemistries may also be used.

In another aspect, an immunotherapy method comprises administering to the subject, e.g., a human subject, a nanoparticle system as described herein. Exemplary human subjects include cancer patients and patients with immune disorders such as multiple sclerosis and rheumatoid arthritis. The nanoparticles can target the immune system by interacting with T cells, cancer cells and/or antigen presenting cells.

When the therapeutic peptides are immune checkpoint inhibitor peptides, the compositions and methods described herein are applicable to all cancers including triple negative breast cancer, head and neck squamous cell carcinoma, melanoma, colorectal cancer, prostate cancer, renal cell cancer, or bladder cancer.

The methods described herein can be further combined with additional cancer therapies such as radiation therapy, chemotherapy, surgery, and combinations thereof.

The invention is further illustrated by the following non-limiting examples.

### EXAMPLES

### Example 1: Synthesis and Preparation of DMs

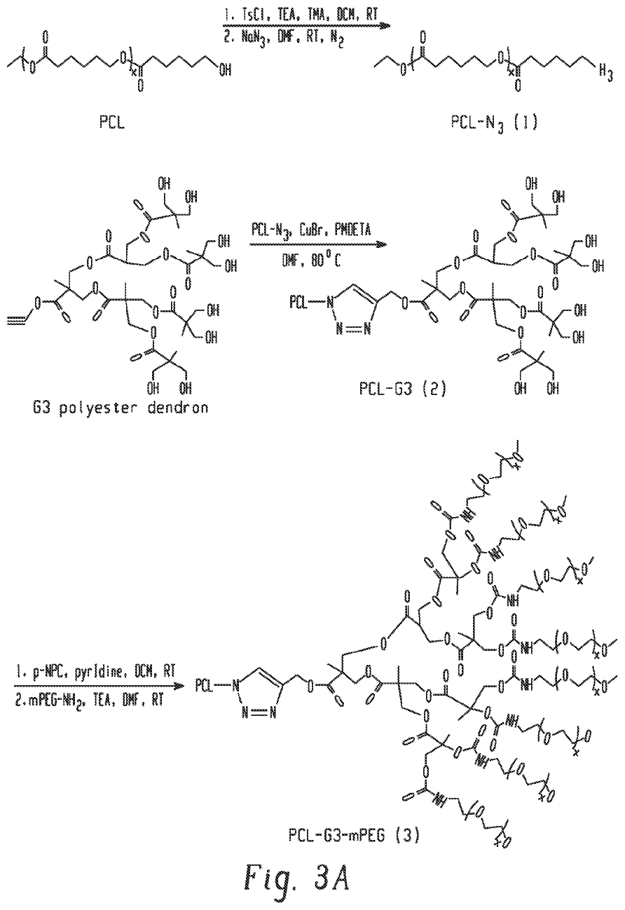

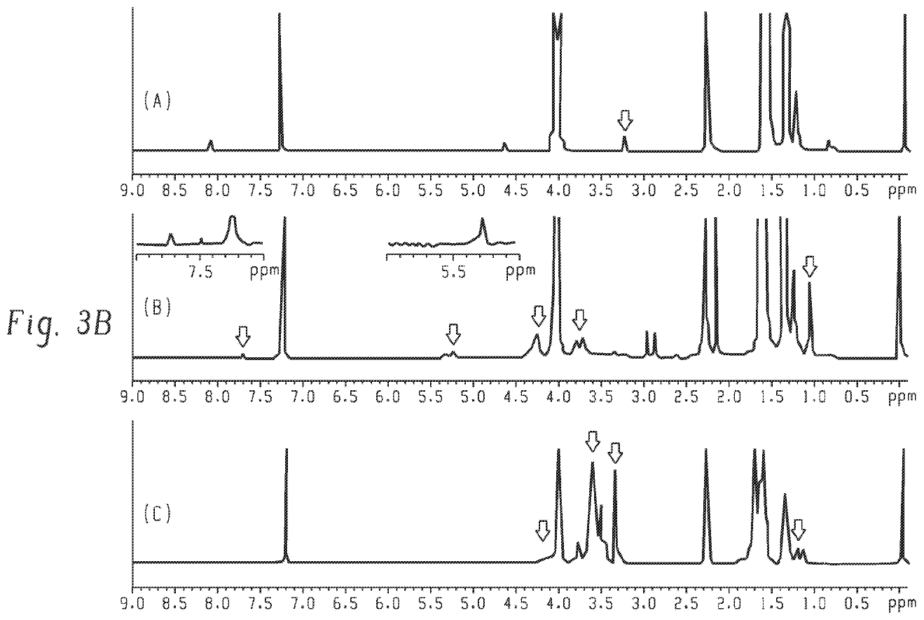

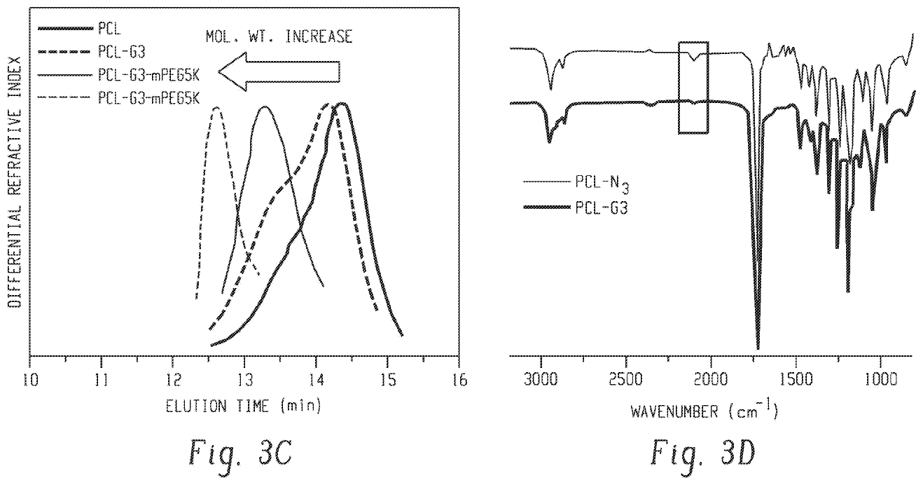

A series of PEGylated dendron coils (PDCs) were designed as a modular drug delivery vehicle, consisting of PCL, G3 dendron, and PEG. Two different molecular weights (MWs) of PCL (3.5 and 14 kDa) and mPEG (2 and 5 kDa) were used to vary the hydrophilic lipophilic balances (HLBs) of the resulting PDCs. The synthetic route and characterization of the PDCs are summarized in FIG. 3. The terminal hydroxyl group of PCL was first converted to an azide group (PCLN3) for subsequent click chemistry with the dendron, as confirmed using ¹H-NMR (FIG. 3B).

The G3 dendron bearing an acetylene group at the focal point was reacted with PCL-N3 via “click chemistry” to yield the PCL-G3 copolymers (FIG. 3B/D). Then, PCL-G3 copolymers were conjugated with methoxyterminated PEG (mPEG), following activation of the surface hydroxyl groups of the dendrons with p-nitrophenyl chloroformate (p-NPC). The MWs and molecular weight distribution (MWD=Mw/Mn, 1.0 meaning perfectly monodisperse) of all intermediate and final products were measured using gel permeation chromatography (GPC) as shown in FIG. 3C. In parallel, the linear copolymer counterparts with the same MW polymers, without dendrons, were also prepared by a similar protocol using p-NPC activation of the hydroxyl group on PCL, followed by mPEG conjugation. All 8 amphiphilic copolymers (4 dendron-based and 4 linear) were successfully synthesized with low MWDs (<1.4).

Functionalized PDCs containing rhodamine as a fluorophore and folic acid (FA) as a model targeting agent were also synthesized. Briefly, PCL14K-G3 was reacted with molar excess (20×) of PEG diamine, resulting in PCL-G3-PEG-NH2. The copolymers were then conjugated with either n-succinimidyl ester (NHS)-functionalized rhodamine (Rho) or FA using chemistry published in the art. Synthesis of the two copolymers were confirmed using ¹H-NMR and UV/Vis, revealing that PCL-G3-PEG-Rho and PCL-G3-PEG-FA contained ˜1 Rho and ˜2 FA molecules per PDC, respectively, as described in the art.

### Example 2: Low CMCs, High PEG Surface Coverage, and Controlled Drug Release of DMs

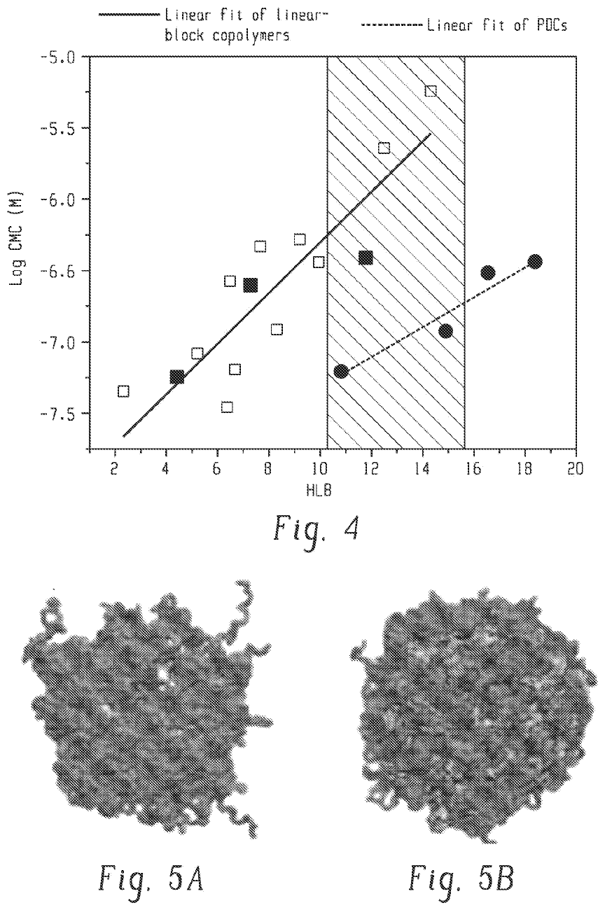

To investigate the self- assembly behaviors of PDCs with various MWs, their critical micelle concentration (CMC) values were measured (FIG. 4) and their self-assembled structure was observed using TEM, which was compared to those of the linear copolymer counterparts. A low CMC is an important requirement for a drug delivery vehicle due to the immediate dilution factor upon injection into the blood stream. FIG. 4 shows nearly linear correlation between CMC and hypophilic lipophilic balances (HLB), observed for both sets of copolymers. Including literature values of CMCs of the linear copolymers composed of the same polymer blocks, it was observed that CMCs of PDCs were 1-2 orders of magnitude lower than those of the linear copolymers at the same HLBs. These data provide solid evidence that the pre-organized molecular architecture of the multiple PEGs and a single PCL chemically combined through a dendron facilitates the formation of remarkably stable PDC self-assemblies with large hydrophilic proportions.

A clear difference between the micelles from PDCs and linear copolymers was also found when the structures were simulated using molecular dynamics (MD) as shown in FIG. 5. The surface of DM (FIG. 5A) is almost fully covered by the PEG layer due to the dendron maximizing the PEG surface density. However, the micelle assembled from linear copolymer has the hydrophobic part being exposed (FIG. 5B). The full surface coverage of a nanocarrier by the PEG layer is critical to take advantage of the “stealth effect” that maximize the circulation time in the body while minimizing non-specific interactions such as reticuloendothelial clearance (RES).

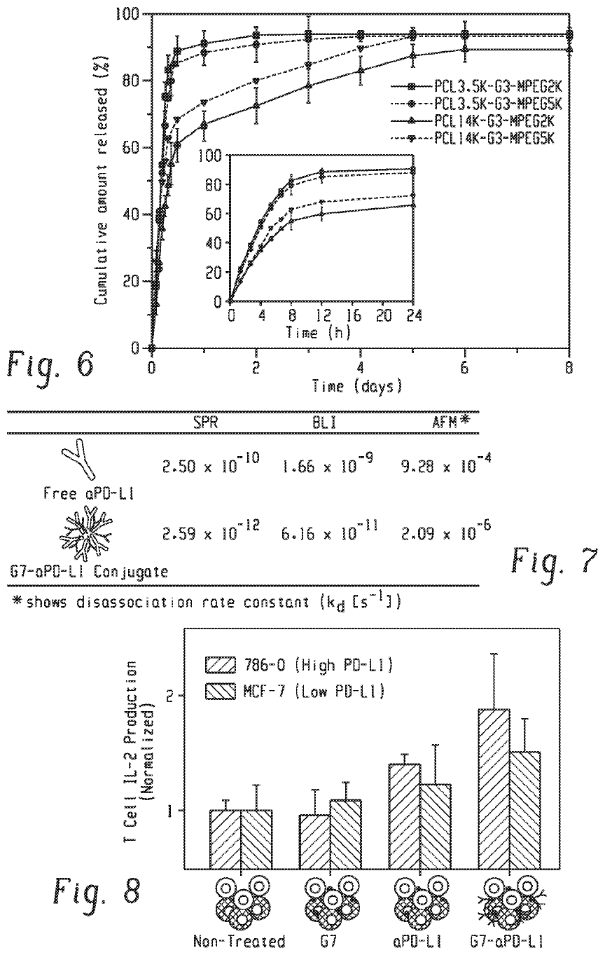

Release profiles of indomethacin (as a model drug) from various DMs were also measured. As shown in FIG. 6, DMs released the drug molecule over 7 days in a sustained manner For the first 12 hrs, the release kinetics were more linear, followed by additional slower release through day 2-8, achieving slow release profiles controlled by MW of PCL.

### Example 3: In vitro/in Vivo Validation of aPD-L1 and aEGFR Delivered by Dendrimers

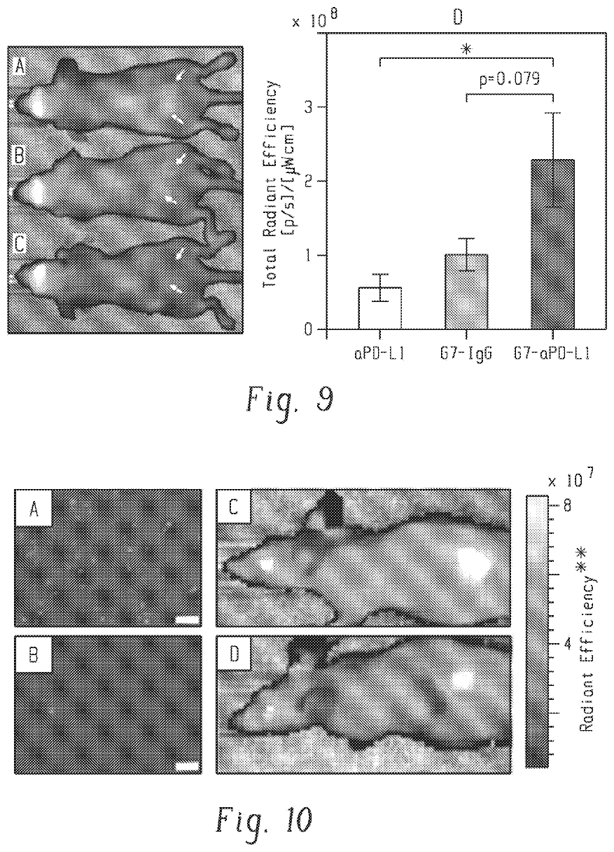

Antibodies that bind to EGFR or PD-L1 were then tested both in vitro and in vivo. For this, generation 7 polyamidoamine (G7 PAMAM) dendrimers were employed to investigate the effect of multivalent binding that can be imposed through the dendritic structure of the DM nanocarriers. G7 PAMAM dendrimers were conjugated with aPD-L1 using a modified protocol from the art. Briefly, ˜50% of primary amine groups were first acetylated using acetic anhydride, followed conjugation with AlexaFluor® 647. The fluorescent-labeled dendrimers were then fully carboxylated by reaction with succinic anhydride, and subsequently conjugated with aPD-L1 at a ratio of dendrimer:aPD-L1 at 1:5. The final G7-aPD-L1 conjugates were analyzed to have approximately 3.8 aPD-L1 per dendrimer molecule. The conjugates were compared to free aPD-L1 in terms of dissociation kinetics (KD) using surface plasmon resonance (SPR), biolayer interferometry (BLI), and atomic force microscopy (AFM) as summarized in FIG. 7. The three independent measurements all revealed that the G7-aPD-L1 conjugates achieved significantly enhanced binding kinetics, compare to free aPD-L1, by two orders of magnitude. The enhanced binding kinetics of the dendrimer conjugates were successfully translated into in vitro results where the highest interleukin-2 (IL-2) secretion from Jurkat T cells was observed when the cells (coincubated with tumor cells—786O vs. MCF-7) were treated with the dendrimer conjugates (FIG. 8). The experiments were performed following previously published experimental conditions in the art. For an in vivo test, BALB/c mice (7- to 8-week old; female) were purchased from Envigo Laboratories (Indianapolis, IN, USA). Mice were injected with TNBC cell line 4T1 (2.0×10⁵ cells). As the tumors grew and reached 300 mm³, either aPD-L1, G7-IgG, or G7-aPD-L1 (all conjugated with AlexaFluor® 647, or AF647) was injected through the tail vein. The concentrations (128 nM, 50 μL) of aPD-L1 and G7-aPD-L1 were determined after normalization based on the fluorescent intensity. The images (FIG. 9) were taken at a 48 h time point post injection. The results clearly show that the dendrimer conjugates (G7-aPD-L1, FIG. 9A) reached the tumor site more preferentially than the free aPD-L1 and G7-IgG (FIG. 9B and C). The quantitative measurement of the fluorescence intensity also indicated that the over a 4-fold higher amount of G7-aPD-L1 was accumulated to the tumor sites than free aPD-L1 (FIG. 9D).

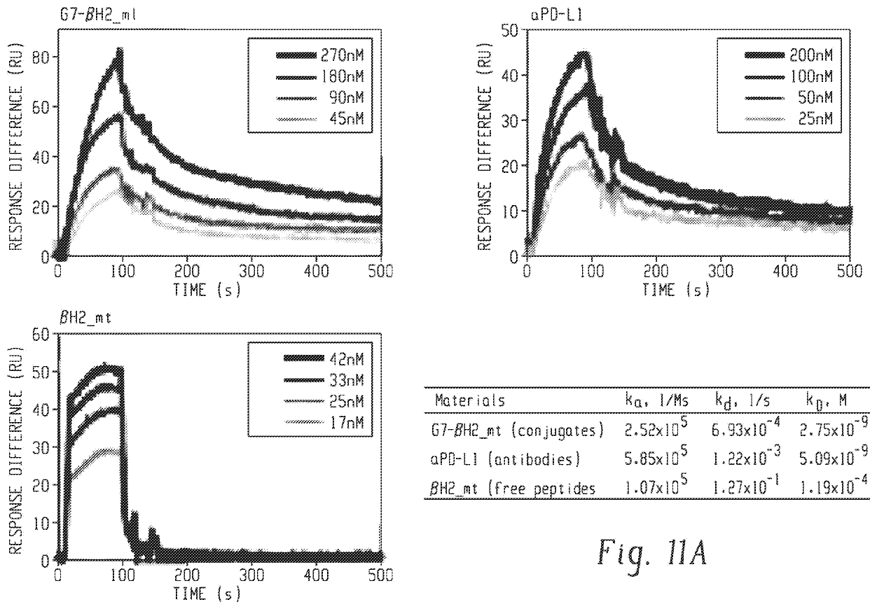

For aEGFR, the resulting conjugates were prepared at a ratio of G7:aEGFR to be approximately 1:10 after conjugation with AF647. The in vitro specificity was measured using MDA-MB-468 that overexpress EGFR. As shown in FIG. 10A/B, G7-aEGFR (10A) exhibited significant interaction with the cells whereas G7 (10B) itself did not, showing that the specificity was directed through aEGFR. In vivo results also revealed the consistent specificity obtained from the G7-aEGFR conjugates (FIG. 10C). Briefly, C57BL/6 mice (7-to 8-week old; female) were acquired from Envigo Laboratories. The mouse TNBC cell line 4T1 (5.0×10⁵ cells) was inoculated into the mice, followed by intravenous injection through tail vein of either free dendrimers or G7-aEGFR.

### Example 4: Peptides to Replace the Antibodies

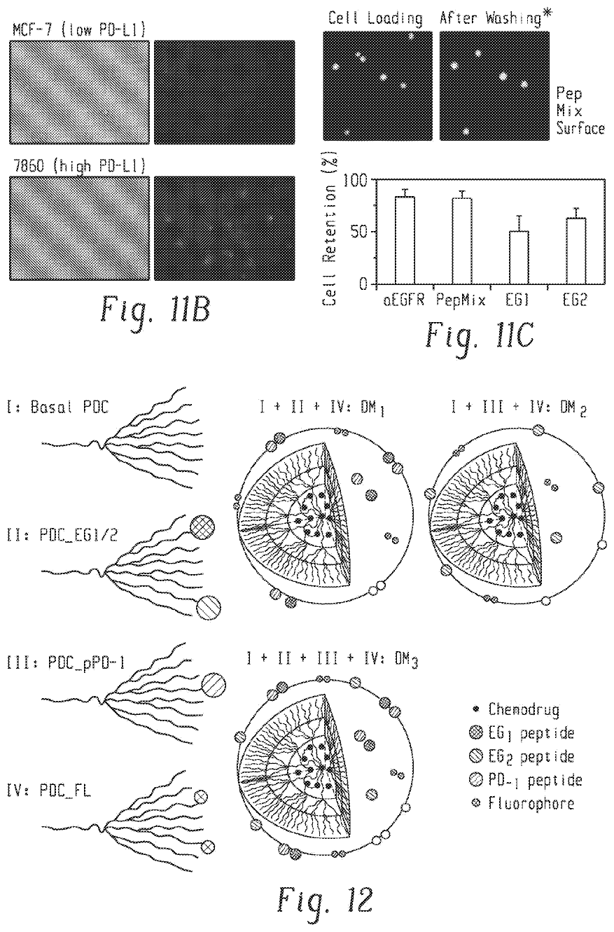

Peptides that bind to EGFR and PD-L1, denoted EG1/EG2 and bH2_mt, respectively, were synthesized according to a protocol from the art. For the PD-L1-binding peptide (bH2_mt), the following peptide sequence: HVVWHRESPSGQTDTLAA SEQ ID NO: 5, optimized from the literature, was used. For EGFR-binding peptides, two peptide sequences were tested: YHWYGYTPQNVI (EG1) SEQ ID NO: 6 and LARLLT (EG2) SEQ ID NO: 7. As noted above, a major challenge of using peptides is their inferior binding kinetics to their corresponding antibodies, despite the advantages stemming from their small MW and flexiblity/capability to be better engineered. It was tested if conjugation of the peptides to PAMAM dendrimers would significantly enhance their binding kinetics through multivalent binding effect, following a previously published protocol. As shown in the SPR results (FIG. 11A), the dendrimer-peptide conjugates (G7-βH2_mt) achieved comparable dissociation constant (KD) to that of full antibody (aPD-L1), which is significantly stronger than free peptide (βH2_mt) by 5 orders of magnitude. The dendrimer-peptide conjugates (FIG. 11B, lower right panel) also displayed a selective binding to PD-L1 expressing cells (786O) as opposed to no apparent interactions with MCF-7 cells with low PD-L1 expression (FIG. 11B, upper right panel). The EGFR-binding peptides were also tested after being surface immobilized in terms of cell retention using EGFR-overexpressing MDA-MB-468 cells. The cells were incubated on the functionalized surfaces with either aEGFR or peptides for 30 min, followed by washing at a shear rate of 25 s⁻¹ for 20 min. As shown in FIG. 11C, the surface with a mixture of EG1 and EG2 showed a similar level of cell retention capability to the whole antibody (aEGFR), indicating that the use of the mixture of the two peptides would achieve stronger binding.

This data demonstrates: i) synthesis of PDCs and their self-assembly into DMs; ii) enhanced selectivity of aPD-L1 and aEGFR; and iii) synthesis of peptides and their significantly enhanced binding kinetics through multivalent binding effect.

### Example 5: Prepare a Series of Functionalized PDCs and Engineer their Binding Kinetics and Self-Assembly into DMs

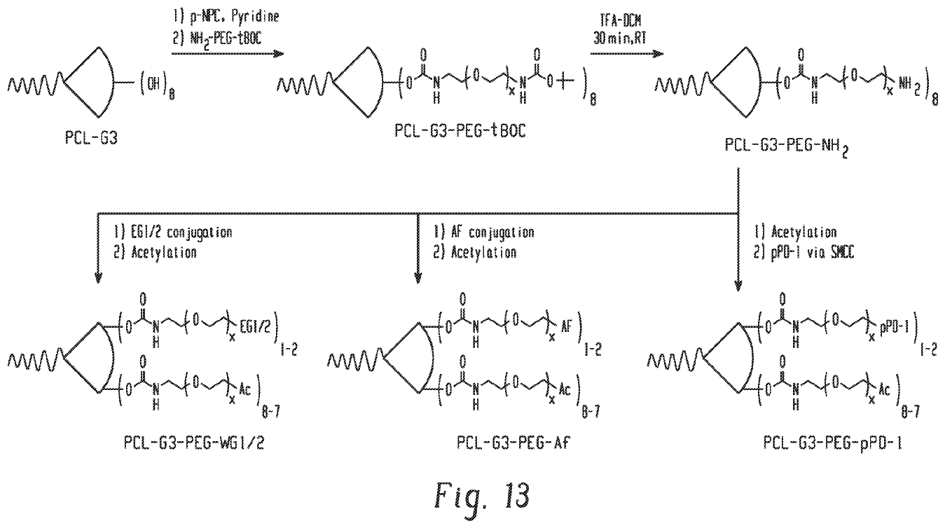

A series of PDCs grouped based upon the number of functionalities will be prepared. The approach will start from the simplest groups and proceed to more complex materials, increasing the likelihood of success of each step of the proposed study. The following PDCs will be synthesized: Acetylated PDC (PCL-G3-PEG-Ac) (I in FIG. 12), EG1/2-conjugated PDC (PCL-G3-PEG-EG1/2) (II in FIG. 12, FIG. 13), PDC conjugated with PD-L1-binding peptides (pPD1) (PCL-G3-PEG-pPD1) (III in FIG. 12, FIG. 13), and PDC with Alexa Fluor® 647 (PCLG3- PEG-AFs) (IV in FIG. 12, FIG. 13).

Selection of MWs of each polymer component of PDCs: The molecular weight (MW) of each polymer block would largely affect the physical and biological properties of the resulting PDCs. Based on the results, the experiments will begin with PCL with 3.5 kDa, PEG with 2 kDa for II and III and 600 Da for I and IV, and G3 dendron (870 Da). The MW of PEG can be important as the tethered configuration aids in maximal specific interaction between the targeting ligand on DMs and cell surface. A previous study revealed that the DMs self-assembled from PEG2K tethered with targeting ligands and PEG0.6K significantly amplified the specific interactions. G3 polyester dendron will be chosen as it is large enough to provide multiple (eight) functional end groups and yet small enough to maintain the MWD at minimum. Depending on initial results, MWs will be varied to control release profiles and the HLBs.

Peptide synthesis: A total of 4 peptide sequences will be synthesized. For PD-L1 binding peptides, in addition to the human sequence used for a recent study, a mouse sequence: IYLCGAISLHPKAKIEESPGA SEQ ID NO: 8 will be prepared for the subsequent in vivo mouse model studies. The same EG1/EG2 peptides will be used for both in vitro and mouse in vivo, given the 89% overlapping between mouse and human These peptides will be synthesized using 9-fluorenylmethoxycarbonyl (Fmoc)-based solid-phase synthesis technique and standard amino acid protecting groups on a Rink Amide MBHA resin LL. After the synthesis, peptides will be cleaved from the resin by 3 h treatment of a cleavage cocktail [trifluoroacetic acid (TFA)/triisopropylsilane (TIS)/water=95:2.5:2.5]. The mixtures will then be precipitated using tert-butyl methyl ether (TBME). The crude peptide solutions will be purified using reverse-phase HPLC with a C18 semi-preparative column. HPLC conditions will be as follows: eluents (solvent A, water with 0.1% TFA; solvent B, acetonitrile with 0.1% TFA), flow rate (2 mL/min), and wavelength for UV detection (230 nm). Molecular weights of the peptides will be confirmed by matrix-assisted laser desorption/ionization time-of-flight mass spectrometry (MALDI-TOF MS), after co-crystallization with α-Cyano-4-hydroxycinnamic acid (CHCA) matrix. Their concentration will be determined by spectrophotometric measurement in water/acetonitrile (1:1) while using molar extinction coefficients of tryptophan (5690 M⁻¹ cm⁻¹) and tyrosine (1280 M⁻¹ cm⁻¹).

Conjugation of PDCs with functional agents: The synthetic routes of functionalized PDCs are illustrated in FIG. 13. Briefly, p-NPC activated PCL-G3 (MW 5,690 Da) will be conjugated with NH2-PEG-tBOC, followed by deprotection with TFA, resulting in PCL-G3-PEG-NH2. The amine groups will provide reactive sites for various bioactive agents such as the EG1/2 and pPD-1 peptides and imaging agents AF647 (AF). Conjugation with the peptides and AF will utilize the same chemistry published in the art. After all the conjugation reactions, the remaining amine groups will be acetylated to protect any potential non-specific interactions. The full acetylation is critical to achieve specific targeting without non-specific interactions. As a control group, linear block copolymers (PCL-PEGs) with the identical MWs with the PDCs will be also prepared to investigate the role of dendrons on the binding and biological behaviors.

DM formation through self-assembly: Various types of PDCs will be self-assembled into micelles, as illustrated in FIG. 12 and FIG. 13. For quantitative fluorescence analyses, the content of PCL-G3-PEG-AF (PDC_FL) will be fixed at 5%, and all other functional components will be mixed at various ratios from minimal (5%) to maximal (30%). For DMs without paclitaxel, 20 mg of PDCs at various ratios will be dissolved in 2 mL of dimethylformamide (DMF). The solution will be dialyzed (MWCO 3.5K) against distilled water for 1 day and freeze dried for 2 days. For encapsulation of paclitaxel, 20 mg of PDCs at the same various ratios will be dissolved in 4 mL of DMF along with 2-4 mg of paclitaxel. The PDC-drug solutions will be then transferred to the dialysis membrane, dialyzed for 24 h against 2 L of distilled water, and freeze dried for 2 days to produce drug-loaded micelles. All the micelles will be characterized in terms of their morphology, size, and surface charge using AFM, TEM, and DLS. CMCs and encapsulation efficiency will be also measured using the methods in the art.

Optimization of EG1/2 and pPD-1 binding kinetics: The mixing ratio of PCL-G3-PEG-EG1/2 (PDC_EG1/2) and PCL-G3-PEG-pPD-1 (PDC_pPD-1) per micelle will be optimized by binding kinetics measured by SPR using BIAcore™ X (GE Healthcare), BLI, AFM (Asylum MFP-3D BioInfinity), according to the method known in the art. Briefly, EGFR and PD-L1 will be immobilized, individually or together, on a substrate, and the binding behaviors of DMs containing various amount of PDC_EG1/2, PDC_pPD-1, or both (5-30% in total content) will be measured. The binding parameters (ka, kd, KA, and KD) will be quantitatively calculated, and the values will be compared to in vitro cell uptake to finalize an optimal ratio of PDC with targeting peptides for a maximal multivalent binding effect.

Preparation and characterization of PDCs and DMs: A large library of PDCs will be established after confirming the chemical structures of all PDCs prepared. PDCs within 10% deviation from theoretical MWs will be used and their molecular weight distributions (MWDs) will be maintained to be below 1.2. The strict threshold both in MWs and MWDs will minimize the batch-to-batch variations and structural heterogeneity of PDCs. The prepared DMs will have a size of ˜50 nm in diameter and contain at least 10 wt % of paclitaxel, along with >90% surface coverage by the PEG outer layers.

Number of functional groups: Based on preliminary studies, it is preferred to maintain the functional groups to b to be less than 3 molecules (peptides) per dendron, in order to maintain the structural regularity of the PDCs.

### Example 6: Validate the Advantages of EGFR Targeting for Chemotherapy In Vitro and In Vivo

In vitro release and selectivity tests will be performed, followed by extensive in vivo study using DM1 (as defined in FIG. 12). Note that the mixture of EG1 and EG2 peptides will be used for the DM1 formulation, based on our preliminary data shown in FIG. 11C, where the EG1/2 mixture exhibited significantly improved cell retention capability than being used separately.

Release kinetics of paclitaxel and in vitro specificity of DMs: DMs containing paclitaxel will be tested in terms of their release kinetics using a dialysis method in the presence of serum, as described in the art. Briefly, 1.5 mL of DMs with paclitaxel (1 mg/mL) will be mixed with 1.5 mL of FBS and placed in a dialysis membrane (MWCO 3.5 kDa) to dialyze against 27 mL of 50% FBS 37° C. with gentle shaking (100 rpm), followed by collection of dialysates at various time points. The paclitaxel content in the collected samples will be quantified by the UV/Vis detection. The in vitro selectivity and cytotoxicity of DM1 will be tested using EGFR positive (4T1, MDA-MB-231, and MDA-MB-468), and EGFR negative (MDA-MB-435 and SUM52) TNBC cell lines, compared to the DMs without the EG1/2 peptide.

In vivo tumor-retention properties of DM1: The goal of this experiment is to determine if the DM1 exhibits longer retention at the tumor. To carry out this experiment, the mouse TNBC cell line 4T1 xenografted onto BALB/c mice will be used. Various DMs will be assembled with AF and paclitaxel to keep the chemistry consistent throughout the in vivo experiments. Empty DMs without paclitaxel are indicated by an asterisk* (DM*). Briefly, 4T1 tumor cells will be prepared and 1.5-2×10⁶ live cells in 100 μL will be injected into the dorsal flank of the mice. Tumors will be measured twice weekly using Vernier calipers and calculated according to the equation V=(π/6) (large diameter)×(small diameter)²; when average tumor volume reaches 100-200 mm³, mice will be randomized into three groups (n=12, single tumors/mouse): 1) no treatment, 2) DM*, 3) DM1. Ten mg/kg of each DM will be delivered in 50-100 μL by tail vein. Primary Endpoint: Tumors will be imaged, using the IVIS Spectrum system (UW Small Animal Imaging Facility), at time 0 h, 6 h, 12 h, 24 h, 3 d, 5 d, and 7 d after injection. Living Image Software from the IVIS Spectrum Series will be used to measure and quantitate total radiant efficiency for uptake and retention.

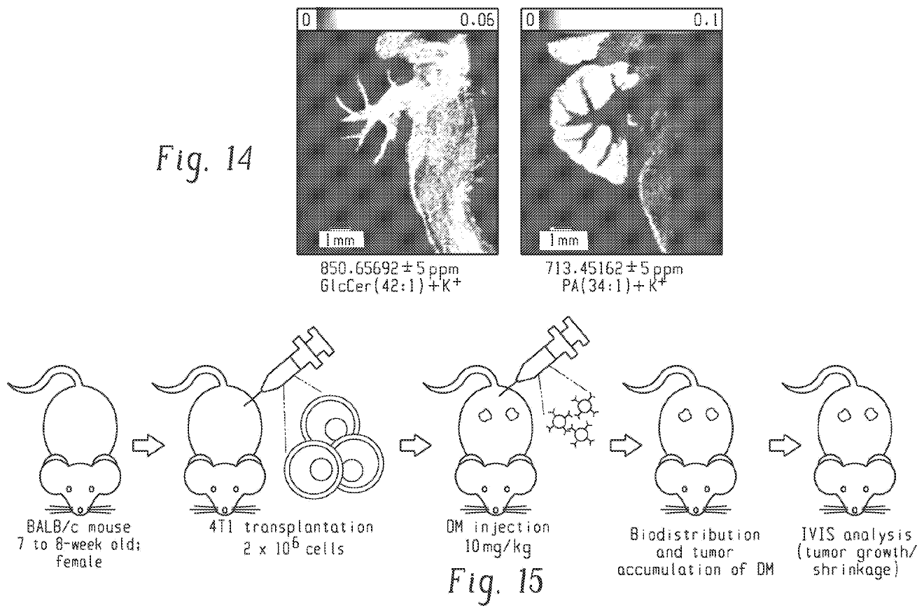

In vivo biodistribution of DM1 and paclitaxel: The goal of this experiment is to determine if DM1 has superior delivery of paclitaxel into the physical tumor versus standard free paclitaxel. The same 4T1-xenografted BALB/c mice will be used. When average tumor volume reaches 100-200 mm³, mice will be randomized into four groups (n=12, single tumors/mouse): 1) no treatment, 2) free paclitaxel, 3) DM w/ paclitaxel, and 4) DM1. 50-100 μL of each DM delivering 22.5 mg/kg of paclitaxel will be delivered by tail vein. To determine the biodistribution and in vivo fates of DM delivery of paclitaxel, matrix-assisted laser desorption ionization-mass spectrometry imaging (MALDI-MSI) will be used, in addition to conventional fluorescence-based techniques. Utilizing MALD-MSI, the proteome of tissue sections can be determined in situ, to generate images depicting differential protein expression in tissue (FIG. 14). MALDI-MSI is label-free and enables simultaneous mapping of numerous molecules in tissue samples with superior sensitivity, quantification and chemical specificity. MALDI-MSI is an unbiased, high-throughput technique that is capable of mapping the spatial distribution of delivered drug compounds, drug metabolites, and possible drug targets due to its high chemical specificity, spatial resolution and sensitivity. Tumors and tissue (blood, liver, lung, brain and kidney) will be collected and fixed accordingly in preparation for MALDI-MSI quantitation analysis.

In vivo efficacy of DM1, compared to free paclitaxel: This experiment is designed to see if paclitaxel released from DM1 can result in better tumor growth control as compared to standard delivery of paclitaxel. The same 4T1-xenografted BALB/c mice will be used. Following inoculation of 4T1 cells, tumors will be measured as described above. Again, when average volume reaches 100-200 mm³ mice will be randomized into five groups (n=16, single tumors/mouse): 1) no treatment, 2) DM*, 3) free paclitaxel, 4) DM with paclitaxel and 5) DM1. The same dose (22.5 mg/kg of paclitaxel) will be delivered by tail vein twice weekly for 4-5 weeks. Primary Endpoint: Tumor growth will be the primary endpoint. Tumors will be measured 3× weekly and plotted followed by statistical analysis for significance.

We anticipate that DM1 will exhibit selective cell interactions (to EGFR+cells only) as well as improved in vivo tumor accumulation and longer retention, compared to non-targeted DM. Furthermore, DM1 will deliver higher concentrations of paclitaxel to the tumor and lower amounts to normal tissue, leading to greater tumor control, compared to free paclitaxel and non-targeted DMs.

### Example 7: Validate the Synergistic Advantages of EGFR and PD-L1 Targeting for Combined Immune and Chemo-Therapies In Vitro and In Vivo

An in vitro confirmation study of selectivity and cytotoxicity of various DMs will be performed, followed by extensive in vivo study using the same mouse model, to compare efficacy of DM1-3.

In vitro selectivity and cytotoxicity of various DMs: Three TNBC cell lines that overexpress PD-L1 and EGFR will be employed, such as MDA-MB-231 and MDA-MB-468, and that express only low levels of PD-L1 and EGFR, such as MDA-MB-435. In vitro specificity of DM2 and DM3 will be confirmed using fluorescence microscopy and flow cytometry, using protocols known in the art. The cytotoxicity of various formulations will be also tested on the cells and measured using enzyme assays, such as LDH and MTT assays, in addition to the microscopic observations.

In vivo tumor-retention properties of DM2 and DM3: The goal of this experiment is to determine if the DM3 exhibits longer retention at the tumor as compared to DM1 and DM2. This experiment will be also carried out using the same 4T1-xenografted BALB/c mice, as illustrated in FIG. 15. Briefly, the same number of 4T1 tumor cells (1.5-2×10⁶ live cells in 100 μL) will be injected into the dorsal flank of the mice. Tumors will be measured until they reach 100-200 mm³. The mice will then be randomized into five groups (n=12, single tumors/mouse): 1) no treatment, 2) DM (without any targeting agents), 3) DM1, 4) DM2, and 5) DM3. Ten mg/kg of each DM will be delivered in 50-100 μL by tail vein. Tumors will be imaged, using the IVIS system, at the time points with the tumor retention experiments described above.

In vivo biodistribution of DM2 and DM3, compared to free paclitaxel: This experiment is designed to determine if DM3 has superior delivery of paclitaxel into the physical tumor versus 1) standard free paclitaxel, 2) DM1 and 3) DM2. The experimental procedures will be identical with those above until the average tumor volume reaches 100-200 mm³. The mice will be randomized into six groups (n=12, single tumors/mouse): 1) no treatment, 2) free paclitaxel, 3) DM, 4) DM1, 5) DM2, and 6) DM3. The same dose of 22.5 mg/kg of paclitaxel will be delivered intravenously. MALDI-MSI will be used for biodistribution and tumor accumulation of paclitaxel delivery via various DMs, in addition to conventional fluorescence-based techniques as described above.

In vivo efficacy of DM3, compared to DM1, DM2, and free paclitaxel: The goal of this experiment is to see if paclitaxel delivery in combination with PD1/PDL1 blockade (DM3) is superior to DM delivered paclitaxel alone (DM1) or DM without EGFR targeting (DM2). The same experimental conditions will be used. When average volume reaches 100-200 mm³ mice will be randomized into six groups (n=16, single tumors/mouse): 1) no treatment, 2) free paclitaxel, 3) DM1, 4) DM2, 5) DM1/DM2 mixture, 6) DM3. Physical mixture of DM1/DM2 will be included in this experiment to see if DM3 integrating all components within a single nanoparticle shows truly synergistic effect. The same dose of paclitaxel will be delivered by tail vein twice weekly for 4-5 weeks. Tumor growth will be the primary endpoint. Tumors will be measured 3× weekly and plotted followed by statistical analysis for significance.

It is expected that DM3 will exhibit selective cell interactions (to EGFR+/PD-L1+cells only) as well as improved in vivo tumor accumulation and longer retention, compared to non-targeted DM, DM1, and DM2. Importantly, significantly increased tumor accumulation, therapeutic index, and overall mouse survival are expected from DM3 compared to other formulations and free paclitaxel. The enhanced results will be attributed to DMs delivering paclitaxel with simultaneous immune checkpoint blockade.

### Example 8: Validate DMs to Treat HNSCC

Similar to Example 5, DMs will be made encapsulating docetaxel instead of paclitaxel. (See, FIGS. 12 and 13). Three HNSCC cell lines that overexpress PD-L1 and EGFR, such as FaDu and MOC1, and that express only low levels of EGFR, such as RPMI2650 will be used. The in vitro specificity of DM1-3 will be confirmed using fluorescence microscopy and flow cytometry. The cytotoxicity of various formulations will be also tested on the cells and measured using enzyme assays, such as LDH and MTT assays, in addition to the microscopic observations.

A series of in vivo experiments will be performed to determine if the DM3 exhibits longer retention at the tumor and enhanced therapeutic efficacy, as compared to DM1 and DM2. This experiment will be carried out using MOC1-xenografted syngeneic BALB/c mice. Briefly, MOC1 tumor cells (1.5-2×10⁶ live cells in 100 μL) will be injected into the dorsal flank of the mice. Tumors will be measured until they reach 100-200 mm³. The mice will be randomized into six groups (n=6, single tumors/mouse): 1) no treatment, 2) free docetaxel, 3) DM1, 4) DM2, 5) DM1/DM2 mixture, and 6) DM3. The numbers of mice and treatment groups will be confirmed after consultation with the SPORE Stats core. The physical mixture of DM1/DM2 will be included in this experiment to see if DM3 integrating all components within a single nanoparticle shows truly synergistic effect. The same dose of docetaxel will be delivered by tail vein twice weekly for 4-5 weeks. Tumor growth will be the primary endpoint. Tumors will be imaged, using the IVIS system, and will be measured 3X weekly and plotted followed by statistical analysis for significance.

A large library of PDCs will be established after confirming the chemical structures of all PDCs prepared. PDCs within 10% deviation from theoretical MWs will be used and their molecular weight distribution (MWDs) will be maintained to be below 1.2. The strict threshold both in MWs and MWDs will minimize the batch-by-batch variations and structural heterogeneity of PDCs. The prepared DMs will have a size of ˜50 nm in diameter and contain at least 10 wt % of docetaxel, along with >90% surface coverage by the PEG outer layers. It is expected that DM3 will exhibit selective cell interactions (to EGFR+/PD-L1+cells only) as well as improved in vivo tumor accumulation and longer retention, compared to non-targeted DM, DM1, DM2, and DM1/DM2 mixture. Importantly, significantly increased tumor accumulation, therapeutic index, and overall mouse survival from DM3 are expected.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A self-assembled immunotherapeutic dendron-micelle, comprising
a first amphiphilic dendron-coil, a second amphiphilic dendron-coil, and a third amphiphilic dendron-coil;
wherein the first amphiphilic dendron-coil comprises a first non-peptidyl, hydrophobic core-forming component covalently linked to a first polyester dendron which is covalently linked to first a poly(ethylene glycol) (PEG) moiety, wherein the first PEG moiety comprises a first conjugated immunotherapeutic peptide;
wherein the second amphiphilic dendron-coil comprises a second non-peptidyl, hydrophobic core-forming component covalently linked to a second polyester dendron which is covalently linked to a second poly(ethylene glycol) (PEG) moiety, wherein the second PEG moiety comprises a second conjugated immunotherapeutic peptide; and
wherein the third amphiphilic dendron-coil comprises a third non-peptidyl, hydrophobic core-forming component covalently linked to a third polyester dendron which is covalently linked to a third poly(ethylene glycol) (PEG) moiety, wherein the third PEG moiety does not comprise a conjugated immunotherapeutic peptide.

2. The self-assembled immunotherapeutic dendron-micelle of claim 1, further comprising an encapsulated chemotherapeutic drug, anti-inflammatory drug, or radiosensitizing molecule.

3. The self-assembled immunotherapeutic dendron-micelle of claim 1, further comprising a fourth amphiphilic dendron-coil comprising a fourth non-peptidyl, hydrophobic core-forming component covalently linked to a fourth polyester dendron which is covalently linked to a fourth poly(ethylene glycol) (PEG) moiety, wherein the fourth PEG moiety comprises a third conjugated immunotherapeutic peptide, an imaging contrast agent, or a chemotherapeutic drug.

4. The self-assembled immunotherapeutic dendron-micelle of claim 1, wherein the first conjugated immunotherapeutic peptide binds a first cell-expressed receptor and the second conjugated immunotherapeutic peptide binds a second cell-expressed receptor, wherein the first and second cell-expressed receptors are on the same or different types of target cells for the immunotherapeutic dendron-micelle.

5. The self-assembled immunotherapeutic dendron-micelle of claim 1, wherein the first and second cell-expressed receptors are selected from immune checkpoint receptors, growth factor receptors, cell surface receptors, and intracellular receptors.

6. The self-assembled immunotherapeutic dendron-micelle of claim 1, wherein the first cell-expressed receptor is an immune checkpoint receptor, and the second cell-expressed receptor is a growth factor receptor.

7. The self-assembled immunotherapeutic dendron-micelle of claim 4, wherein the immune checkpoint receptor comprises PD-L1, PD-1, OX40, TIGIT, CTLA-4, CD137 (4-1BB), CD28, or CD27.

8. The self-assembled immunotherapeutic dendron-micelle of claim 4, wherein the growth factor receptor comprises epidermal growth factor receptor (EGFR), insulin-like growth factor receptor (IGFR), transforming growth factor-beta receptor (TGF-βR), human epidermal growth factor receptor 2 (HER2), vascular endothelial growth factor receptor (VEGFR), platelet-derived growth factor receptor (PDGFR), or fibroblast growth factor receptor (FGFR).

9. The self-assembled immunotherapeutic dendron-micelle of claim 1, wherein the first and second non-peptidyl, hydrophobic core-forming components are selected from polycaprolactone (PCL), poly(lactic acid) (PLA), poly(glycolic acid) (PGA), and poly(lactic-co-glycolic acid) (PLGA), specifically poly(ε-caprolactone).

10. The self-assembled immunotherapeutic dendron-micelle of claim 1, wherein the first and second non-peptidyl, hydrophobic core-forming components have a molecular weight of 0.5 kDa to about 20 kDa, wherein the molecular weights of the first and second non-peptidyl, hydrophobic core-forming components are the same or different, preferably different.

11. The self-assembled immunotherapeutic dendron-micelle of claim 1, wherein the first and second polyester dendrons comprise a generation 3 to generation 5 dendron with an acetylene or carboxylate core, specifically a generation 3 polyester-8-hydroxyl-1-acetylene bis-MPA dendron.

12. The self-assembled immunotherapeutic dendron-micelle of claim 1, wherein the first and second PEG moiety independently comprise a methoxy PEG (mPEG) moiety, amine-terminated PEG (PEG-NH₂) moiety, acetylated PEG (PEG-Ac) moiety, carboxylated PEG (PEG-COOH) moiety, thiol-terminated PEG (PEG-SH) moiety, N-hydroxysuccinimide-activated PEG (PEG-NHS) moiety, NH₂-PEG-NH₂ moiety, or an NH₂-PEG-COOH moiety.

13. The self-assembled immunotherapeutic dendron-micelle any of claim 1, wherein the first and second PEG moiety each have a molecular weight of about 0.2 kDa to about 5 kDa.

14. The self-assembled immunotherapeutic dendron-micelle of claim 1, further comprising a ligand, such as a cancer-cell binding ligand (e.g., folic acid, luteinizing hormone-releasing hormone, a retinoid, transferrin, RGD peptide, Herceptin, prostate-specific membrane antigen (PSMA)-targeting aptamers, follicle stimulating hormone (FSH), epidermal growth factor (EGF), a lectin or an antibody), or an imaging agent, or radiosensitizing molecule.

15. A pharmaceutical composition comprising the self-assembled immunotherapeutic dendron-micelle of claim 1 and a pharmaceutically acceptable excipient.

16. A method of making a self-assembled immunotherapeutic dendron-micelle, comprising
synthesizing a first amphiphilic dendron-coil by covalently linking a first non-peptidyl, hydrophobic core-forming component to a first polyester dendron, covalently linking the first polyester dendron to a first poly(ethylene glycol) (PEG) moiety, and conjugating a first therapeutic peptide to the first PEG moiety;
synthesizing a second amphiphilic dendron-coil by covalently linking a second non-peptidyl, hydrophobic core-forming component to a second polyester dendron, covalently linking the second polyester dendron to a second poly(ethylene glycol) (PEG) moiety, and conjugating a second therapeutic peptide to the second PEG moiety;
synthesizing a third amphiphilic dendron-coil by covalently linking a third non-peptidyl, hydrophobic core-forming component to a third polyester dendron, covalently linking the third polyester dendron to a third poly(ethylene glycol) (PEG) moiety, wherein the third PEG moiety does not comprise a conjugated immunotherapeutic peptide; and
incubating the first, second, and optionally third amphiphilic dendron-coils under conditions for self-assembly of the self-assembled immunotherapeutic dendron-micelle.

17. The method of claim 16, wherein the first and second amphiphilic dendron-coils comprise 5 to 80 wt % of the self-assembled immunotherapeutic dendron-micelle, and the third amphiphilic dendron-coil comprises 20 to 95 wt % of the self-assembled immunotherapeutic dendron-micelle.

18. An immunotherapy method comprising administering a therapeutically effective amount of the self-assembled immunotherapeutic dendron-micelle of claim 1 to a subject in need thereof.

19. The method of claim 18, wherein the subject is in need of treatment for cancer and the cancer is triple negative breast cancer, head and neck squamous cell carcinoma, melanoma, colorectal cancer, prostate cancer, renal cell cancer, or bladder cancer.


SEQUENCE LISTING (7 sequences)

SEQ ID NO: 1 - protein, 21 aa, Artificial sequence
  Beta H1- wt sequence
TYLCGAISLAPKLQIKESLRA

SEQ ID NO: 2 - protein, 21 aa, Artificial sequence
  betaH1- mutant sequence
TYVCGVISLAPRIQIKESLRA

SEQ ID NO: 3 - protein, 18 aa, Artificial sequence
  BetaH2- wild type sequence
VLNWYRMSPSNQTDRKAA

SEQ ID NO: 4 - protein, 18 aa, Artificial sequence
  betaH2 - mutant sequence
HVVWHRESPSGQTDTKAA

SEQ ID NO: 5 - protein, 12 aa, Artificial sequence
  EGFR-binding peptide
YHWYGYTPQNVI

SEQ ID NO: 6 - protein, 6 aa, Artificial sequence
  EGFR-binding peptide
LARLLT

SEQ ID NO: 7 - protein, 21 aa, Artificial sequence
  mouse PD-L1 binding sequence
IYLCGAISLHPKAKIEESPGA